(19) United States
(12) Patent Application Publication
(10) Pub. No.: US 2013/0178410 A1
(43) Pub. Date: Jul. 11, 2013

(54) METHODS FOR MODULATING SLOW MYOSIN

(71) Applicant: NATIONAL TAIWAN UNIVERSITY, Taipei City (TW)

(72) Inventors: Show-Li Chen, Taipei (TW); HSIN-HSIUNG CHEN, Taipei (TW); Szu-Wei Chang, Taipei (TW); JIM PAN, Taipei (TW); Kuan-Liang Lin, Taipei (TW)

(73) Assignee: NATIONAL TAIWAN UNIVERSITY, Taipei City (TW)

(21) Appl. No.: 13/762,821

(22) Filed: Feb. 8, 2013

Related U.S. Application Data
(63) Continuation-in-part of application No. 12/882,546, filed on Sep. 15, 2010.

(51) Int. Cl. A61K 38/17

(52) U.S. Cl. CPC A61K 38/1709
USPC 514/1.1; 514/44 R

(57) ABSTRACT

The present invention provides a method for modulating an expression level of a gene encoding slow myosin in a subject in need thereof, comprising administering to said subject a pharmaceutically effective amount of a nuclear receptor interaction protein (NRIP) and a pharmaceutically acceptable carrier. The present invention also provides a method for modulating an expression level of a gene encoding slow myosin in a subject in need thereof, comprising administering to said subject a pharmaceutically effective amount of an expression vector comprising a gene encoding a nuclear receptor interaction protein (NRIP) and a pharmaceutically acceptable carrier. In a preferred embodiment, the expression vector is an adenoviral vector.

### CROSS-REFERENCE TO RELATED APPLICATIONS

This application is a Continuation-in-part of the pending U.S. patent application Ser. No. 12/882,546 filed on Sep. 15, 2010, for which priority is claimed and is incorporated herein by reference in its entirety.

Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of the prior art of record and any search that the Office deems appropriate.

### FIELD OF THE INVENTION

The present invention relates a method for modulating an expression level of a gene encoding slow myosin in a subject in need thereof, comprising administering to said subject a pharmaceutically effective amount of a nuclear receptor interaction protein (NRIP) and a pharmaceutically acceptable carrier. The present invention also relates a method for modulating an expression level of a gene encoding slow myosin in a subject in need thereof, comprising administrating to said subject a pharmaceutically effective amount of an expression vector comprising a gene encoding a nuclear receptor interaction protein (NRIP) and a pharmaceutically acceptable carrier.

### DESCRIPTION OF PRIOR ART

The muscular dystrophies are a group of clinically and genetically heterogeneous disorders of the skeletal muscle inherited in either autosomal dominant or recessive fashion. Muscular dystrophies are characterized clinically by progressive muscle weakness predominantly in the pelvic and shoulder-girdle muscles, serum creatine kinase (SCK) elevation, normal intelligence and great variability, ranging from severe forms with onset in the first decade and rapid progression to milder forms with later onset and a slower course (Tsai, T. C. et al, J. Biol. Chem., 2005, 280, 20000-20009). The diagnosis of muscular dystrophies can be excluded by the finding of severely abnormal dystrophin staining on muscle biopsies. Although analysis of the defective proteins has shed some light onto their functions implicated in the etiology of muscular dystrophies, our understanding of the molecular mechanisms underlying muscular dystrophy remains incomplete.

Skeletal muscles are a mosaic of slow and fast twitch myofibers. Calcium (Ca²⁺) plays a key role in skeletal muscle contraction both in slow and fast fibers and regulates myosin heavy chain isoforms' gene expression. Now, slow myosin fiber is clearly reportedly regulated by the increased intracellular Ca²⁺. Additionally, testosterone increases the intracellular Ca²⁺ level. Nuclear receptor interaction protein (NRIP) is a transcription cofactor, it contains 860 amino acids and seven copies of WD40 domains, and its expression is restricted to the cell nucleus. NRIP is an androgen receptor (AR)-interacting protein to enhance AR-mediated gene expression, it plays a feed-forward role in enhancing the AR-driven NRIP promoter activity via stabilization of the AR protein (Pei-Hong Chen et al, Nucleic Acids Research, 2008, Vol. 36, No. 1 51-66). NRIP enhances transcriptional activity of either AR or GR (glucocorticoid receptors) via ligand-dependent interactions (Tsai, T. C. et al, J. Biol. Chem., 2005, 280, 20000-20009).

In the recent report, the clinical gene expression profiles of muscular dystrophy patients lack NRIP gene expression by microarray assay. According to the analysis of differentially expressed genes between relative normal and dystrophic muscles from the same Limb-girdle muscular dystrophy (LGMD) patient, NRIP expression pattern was down-regulated in the muscular dystrophy patient (Yong Zhang et al, Journal of Translational Medicine, 2006, 4:53). However, the relation of NRIP caused muscular dystrophy needs to be further investigated.

### BRIEF DESCRIPTION OF THE DRAWINGS

FIGS. 1A-1D show that NRIP binds calmodulin in vivo and in vitro.

FIG. 1A shows that the IQ domain (SEQ ID NO: 6) of NRIP protein (SEQ ID NO: 5) locates on amino acid 691 to 713. The arrows indicate the highly conserved positions of amino acid compared with the other proteins containing IQ domain reported previously. The internal IQ-deleted mutant form of NRIP is generated by site-directed mutagenesis; and named NRIPΔIQ.

FIG. 1B shows that NRIP interacts with Ca²⁺/CaM in vitro. The NRIP proteins from in vitro translation (upper panel) or bacterially expressed (His-NRIP, lower panel) are incubated with CaM-agarose in the buffer containing calcium ions or EGTA. The proteins binding to CaM are then eluted by using EGTA-containing buffer and analyzed with anti-NRIP antibody. The data indicate that NRIP binds to CaM in the presence of calcium.

FIG. 1C shows that IQ domain of NRIP is responsible for Ca²⁺/CaM binding. The equal amounts of in vitro translated wild-type (WT) NRIP and IQ-deleted NRIP proteins of NRIPΔIQ are incubated with CaM-agarose. The CaM-binding proteins are then analyzed by western-blotting with anti-NRIP antibody.

FIG. 1D shows that NRIP interacts with Ca²⁺/CaM in vivo. The 293T cells are transiently co-transfected with NRIP-FLAG and CaM conjugates with EGFP expression plasmids. After 48 h, the cell lysates are collected and immunoprecipitated with anti-FLAG or anti-EGFP for NRIP and CaM, respectively. The immunoprecipitated proteins are then subjected to western-blotting with antibodies indicated.

FIGS. 2A-2E show generation of NRIP knockout mice.

FIG. 2A shows schematic illustration of genomic structure of the NRIP wild-type, NRIP flox, and NRIP-deleted alleles.

FIG. 2B shows southern blot hybridization of mouse tail genomic DNA isolated from wild-type (+/+) and heterozygous (+/−) offspring of heterozygous intercross. After restriction enzyme Sca I digestion and DNA denaturation, the genomic DNA is hybridized by 5′ flanking probe designed on NRIP intron 1 region. The wild-type allele represents a band on the size of 13.27 kb and the NRIP knockout allele represents a band on the size of 11.3 kb.

FIG. 2C shows genome typing of mouse tail DNA from wild-type (+/+), heterozygous (+/−) and homozygous (−/−) offspring by PCR analysis. The result shows a targeted product of 0.7 kb detected by AU-XD primers, and a wild-type product of 0.6 kb detected by KU-XD primers (*: nonspecific band).

FIG. 2D shows expression of NRIP mRNA level in NRIP knockout mice by RT-PCR analysis. The upper panel shows the schematic illustration of the designed primers to detect the deletion of NRIP exon 2; the lower panel shows RT-PCR analysis of NRIP mRNA isolated from testis, heart and skeletal muscle of wild-type (WT) and knockout (KO) offspring. β-actin or GAPDH is examined as a loading control.

FIG. 2E shows expression of mouse NRIP protein in wild-type (WT) and knockout (KO) adult tissues. Following tissue dissection and protein extraction, expression of NRIP is analyzed by Western blot with primary NRIP antibody. The size of NRIP protein is examined by knockdown of NRIP expression in LNCap human prostate cancer cell line (as a positive control). GAPDH is examined as a loading control. The left panel shows the expression of NRIP in WT and KO skeletal muscle tissue; the right panel shows the expression of NRIP and androgen receptor (AR) in WT and KO testis tissue.

FIGS. 3A-3B show expression of NRIP and slow myosin in skeletal muscle tissues of adult male mice (Following the tissue dissection and protein extraction).

FIG. 3A shows western blot analysis of NRIP expression, using total protein (100 μg) from the hindlimb skeletal muscle tissues of adult (10-week) male mice.

FIG. 3B shows analysis of slow myosin (MHC7) expression in soleus and gastrocnomius (Gast.) muscle tissues respectively.

The size of NRIP protein is examined by knockdown of NRIP expression in LNCap human prostate cancer cell line. The GAPDH serves as an internal control for protein loading.

FIG. 4 shows RNA expression of slow myosin in soleus muscle tissues. As described tissues from FIG. 3, RNA is extracted and analyzed for the gene expression of slow myosin (MHC7).

FIG. 5 shows immunohistochemistry analysis of slow myosin expression in gastrocnomius skeletal muscle tissue of 12-week old NRIP^{+/+} and NRIP^{-/-} mice. Following tissue dissection and paraffin embedding, the 4 μm sections are incubated with slow myosin primary antibody (MHC 7) for overnight and stained with 3,3′ Diaminobenzidine (DAB) chromogen. In wild-type mice (A and B), the slow myosin is expressed dispersedly in gastrocnomius tissue. In NRIP^{-/-} mice (C and D), the slow myosin is less expressed in this tissue. (magnification: A and C×100; B and D×200). Arrow mark: slow myosin.

FIG. 6A shows the result of overexpression or downregulation of NRIP that affects protein expression of slow myosin in C2C12 myotube by Western blot analysis.

FIG. 6B shows the result of quantified analysis of protein expression of slow myosin which is affected by overexpression or downregulation of NRIP.

### SUMMARY OF THE INVENTION

The present invention is directed to a method for modulating an expression level of a gene encoding slow myosin in a subject in need thereof, comprising administering to said subject a pharmaceutically effective amount of a nuclear receptor interaction protein (NRIP) and a pharmaceutically acceptable carrier.

The present invention also is directed to a method for modulating an expression level of a gene encoding slow myosin in a subject in need thereof, comprising administering to said subject a pharmaceutically effective amount of an expression vector comprising a gene encoding a nuclear receptor interaction protein (NRIP) and a pharmaceutically acceptable carrier.

### DETAILED DESCRIPTION OF THE INVENTION

The present invention showed that nuclear receptor interaction protein (NRIP) is Ca²⁺-dependent calmodulin binding protein. Moreover, preliminary results of the present invention from NRIP knock out mice model demonstrates the slow myosin protein and RNA expression are declined in NRIP KO mice. The present invention also modulated the expression level of NRIP in a cell by viral vector to demonstrate that the expression of slow myosin is regulated by NRIP. Therefore, NRIP may be involved in skeletal muscle development and be a diagnosis marker and therapeutic target of muscular dystrophy.

The present invention is directed to a method for modulating the expression level of slow myosin comprising administering to a subject in need thereof a therapeutically effective amount of nuclear receptor interaction protein (NRIP) modulator and calmodulin, and a pharmaceutically acceptable carrier. The nuclear receptor interaction protein (NRIP) binds with the calmodulin. And the expression level of slow myosin is protein expression level or RNA expression level. The pharmaceutical composition of the present invention treats skeletal muscle dystrophy.

As used herein in the specification, “a” or “an” may mean one or more. As used herein in the claim(s), when used in conjunction with the word “comprising”, the words “a” or “an” may mean one or more than one.

The present invention provides a method for modulating an expression level of a gene encoding slow myosin in a subject in need thereof, comprising administering to said subject a pharmaceutically effective amount of a nuclear receptor interaction protein (NRIP) and a pharmaceutically acceptable carrier. In one embodiment, NRIP interacts with a calmodulin. In another embodiment, the expression level of a gene encoding slow myosin is DNA, RNA or protein expression level of slow myosin.

In one embodiment, the subject is an animal. Preferably, the subject is a mammal More preferably, the subject is a human.

The present invention may be used to treat, alleviate, ameliorate, relieve, delay onset of, inhibit progression of, reduce severity of, and/or reduce incidence of one or more symptoms or features of a disease, disorder, and/or condition induced by the slow myosin. In a preferred embodiment, the method of the present invention further treats muscular dystrophy.

As used herein, “NRIP” refers to a protein or a gene encoding the protein. In one embodiment, the gene encoding NRIP is SEQ ID NO: 1. In another embodiment, the protein sequence of NRIP is SEQ ID NO: 5.

A “pharmaceutically effective amount” is an amount effective to prevent, lower, stop or reverse the development of, or to partially or totally alleviate the existing symptoms of a particular condition for which the subject being treated. Determination of a therapeutically effective amount is well within the capability of those skilled in the art.

The composition comprising the NRIP can be administered to the subject by many routes and in many regimens that will be well known to those in the art. In some embodiments, the NRIP is administered intravenously, intramuscularly, subcutaneously, topically, orally, or by inhalation. Through the digestive system and circulatory system, it will be delivered to target locations.

The composition comprising the NRIP may be formulated for administering via sterile aqueous solution or dispersion, aqueous suspension, oil emulsion, water in oil emulsion, site-specific emulsion, long-residence emulsion, sticky-emulsion, microemulsion, nanoemulsion, liposomes, microparticles, microspheres, nanospheres, nanoparticles, minipumps, and with various natural or synthetic polymers that allow for sustained release. The compounds comprising the NRIP may also be formulated into aerosols, tablets, pills, sterile powders, suppositories, lotions, creams, ointments, pastes, gels, hydrogels, sustained-delivery devices, or other formulations used in drug delivery.

The pharmaceutically acceptable carriers are determined in part by the particular composition being administered, as well as by particular method used to administer the composition. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a subject. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The present invention also provides a method for modulating an expression level of a gene encoding slow myosin in a subject in need thereof, comprising administering to said subject a pharmaceutically effective amount of an expression vector comprising a gene encoding a nuclear receptor interaction protein (NRIP) and a pharmaceutically acceptable carrier. In one embodiment, the expression vector is an adenoviral vector. In another embodiment, the gene is SEQ ID NO: 1. In still another embodiment, NRIP interacts with a calmodulin. In further embodiment, the expression level of a gene encoding slow myosin is DNA, RNA or protein expression level of slow myosin.

In one embodiment, the subject is an animal. Preferably, the subject is a mammal. More preferably, the subject is a human.

In some embodiment, the present invention can be applied to gene therapy. The expression vector of the present invention can comprise a gene encoding the NRIP, the gene can be administered in vivo to promote expression of its encoded protein, by constructing it as part of an appropriate nucleic acid expression vector and administering it so that it becomes intracellular, e.g., by use of a retroviral vector, or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus, etc. Alternatively, a nucleic acid can be introduced intracellularly and incorporated within host cell DNA for expression, by homologous recombination.

The term “expression vector”, as used here, is meant to include any type of genetic vector containing a polynucleotide sequence coding for a NRIP gene product in which part or all of the NRIP nucleic acid is capable of being transcribed and subsequently translated into a protein.

As referred to herein, the term “encoding” is intended to mean that the gene or nucleic acid may be transcribed in a cell, e.g., when the nucleic acid is linked to appropriate control sequences such as a promoter in a suitable vector (e.g., an expression vector) and the vector is introduced into a cell. Such control sequences are well known to those skilled in the art.

As used herein, the term “gene” means a nucleic acid which encodes a protein or functional fragment thereof. The term “nucleic acid” is intended to mean natural and synthetic linear and sequential arrays of nucleotides and nucleosides, e.g., in cDNA, genomic DNA (gDNA), mRNA, and RNA, oligonucleotides, oligonucleosides and derivatives thereof. It will also be appreciated that such nucleic acids can be incorporated into other nucleic acid chains referred to as “vectors” by recombinant-DNA techniques such as cleavage and ligation procedures.

### EXAMPLES

The examples below are non-limiting and are merely representative of various aspects and features of the present invention.

### Example 1

### NRIP Binds Calmodulin In Vitro and In Vivo

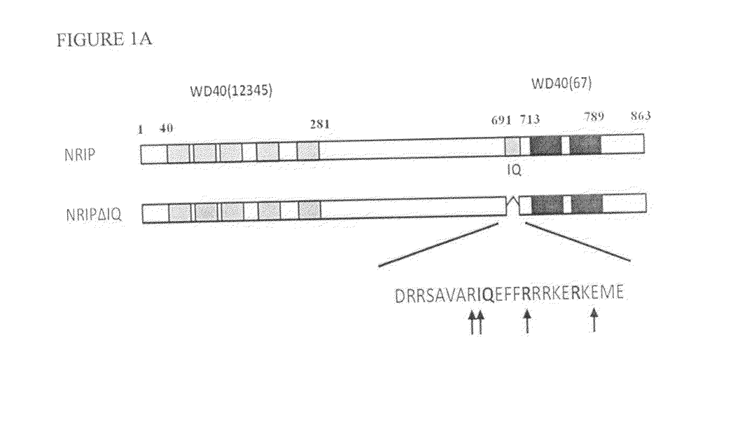

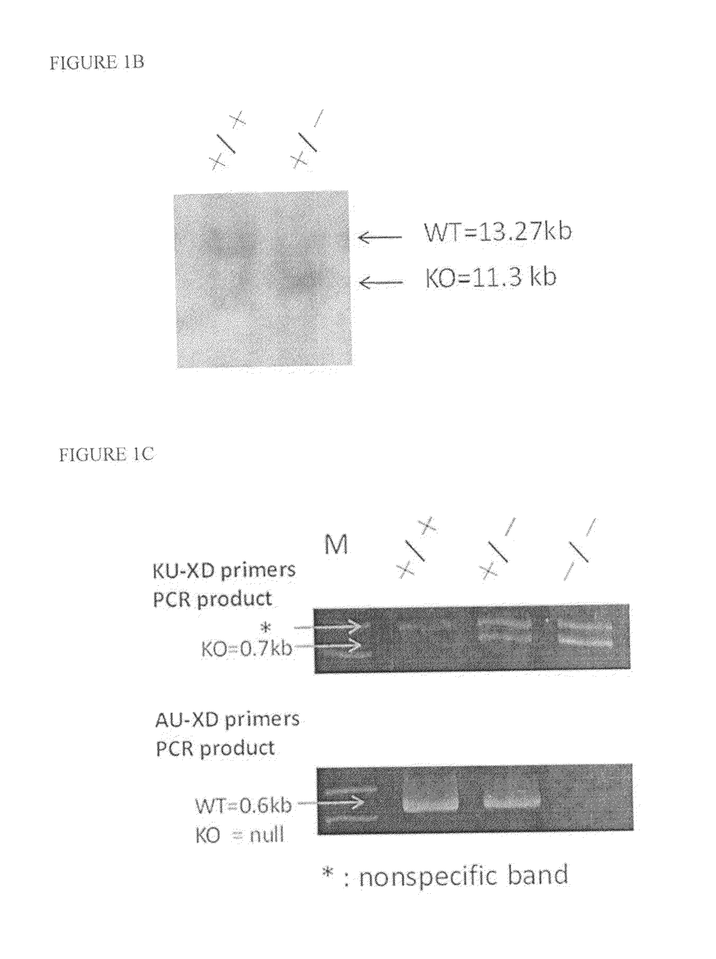

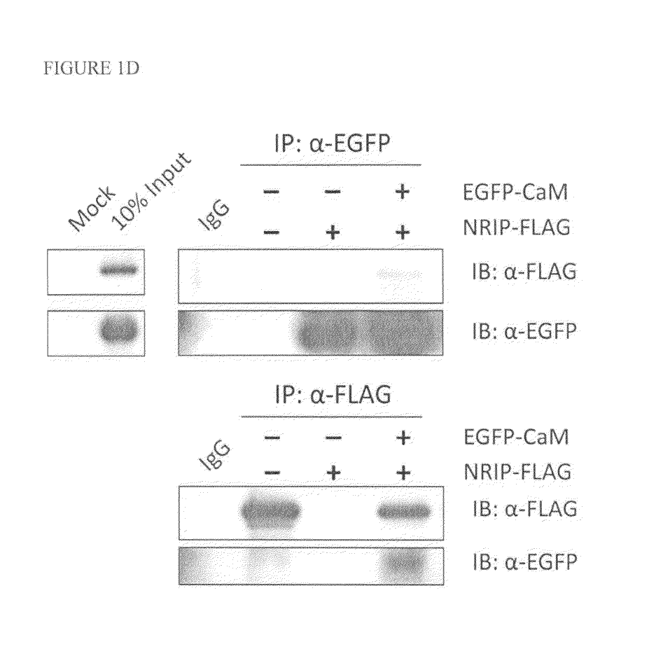

The wild-type NRIP protein sequence (SEQ ID NO: 5) and IQ domain (SEQ ID NO: 6)-deleted NRIP proteins from in vitro translation or bacterially expressed His-NRIP were incubated with CaM-agarose. The proteins bound to CaM were then eluted by using EGTA-containing buffer and analyzed with anti-NRIP antibody. These data indicated that NRIP bound to CaM in the presence of calcium (FIG. 1B and FIG. 1C). To test the NRIP that could interact with CaM in vivo, the 293T cells were transiently co-transfected with NRIP-FLAG and CaM conjugated with EGFP expression plasmids. After 48 h, the cell lysates immunoprecipitated with anti-FLAG or anti-EGFP for NRIP and CaM, respectively and then analyzed with immunoblot (FIG. 1D). The results showed that NRIP interacts with CaM.

### Example 2

### Generation of NRIP Knockout Mice

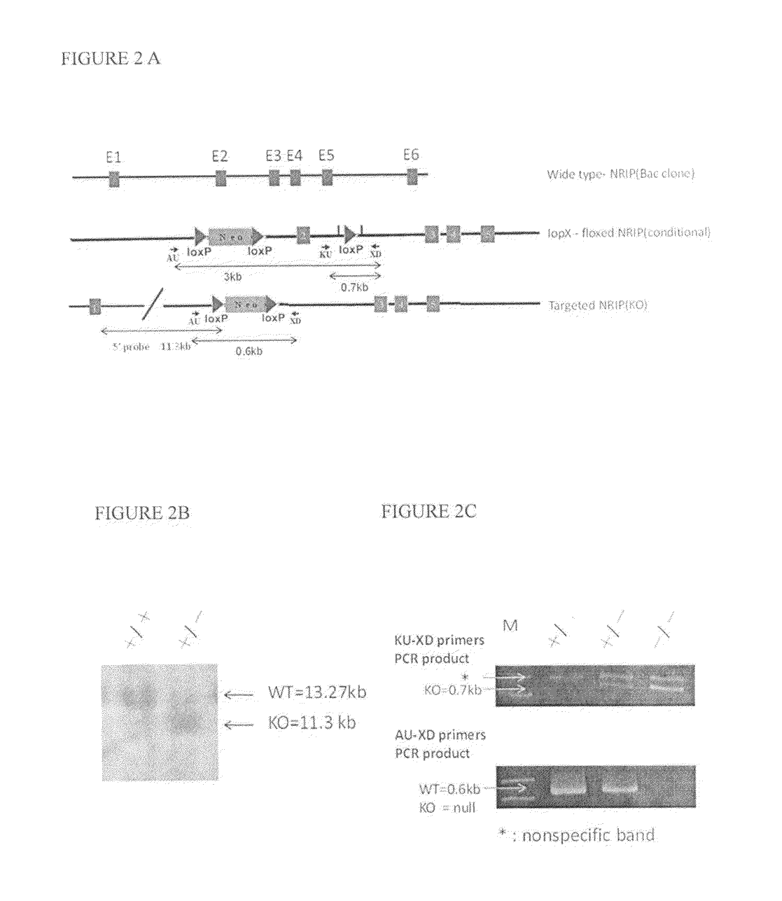

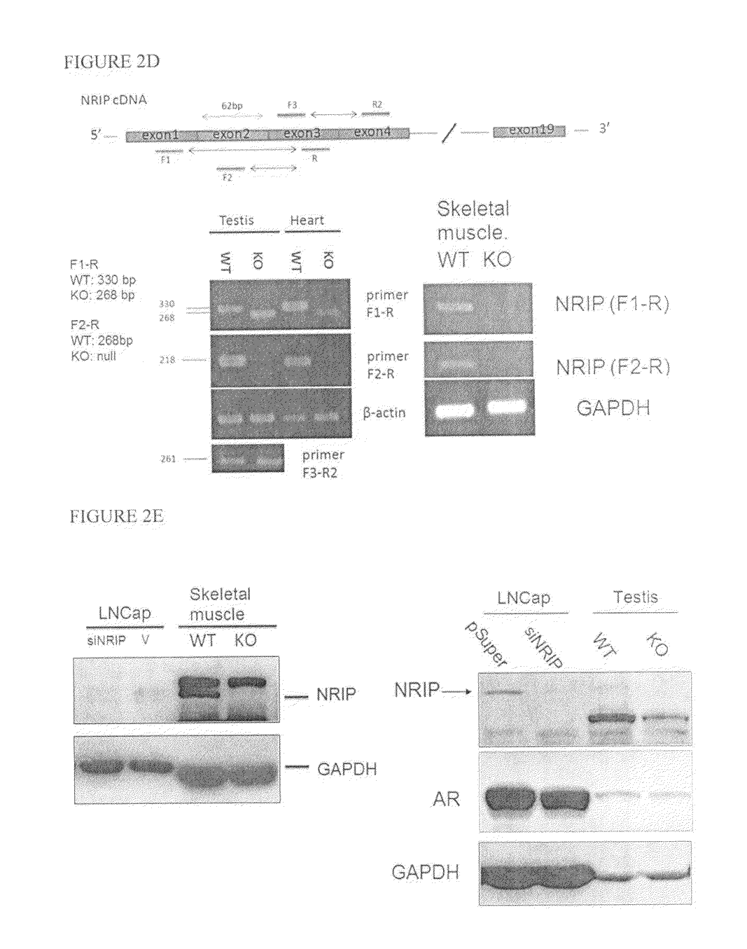

The loxP-floxed NRIP conventional knockout mice were suitable for investigating the role of NRIP in skeletal muscle development. The NRIP exon 2 was deleted after loxP site recombination (FIG. 2A). The genome NRIP deletion was confirm by Southern blot (FIG. 2B) and the present invention designed three primers consisting of AU primer (SEQ ID NO: 2), KU primer (SEQ ID NO: 3) and XD primer (SEQ ID NO: 4) to detect mouse tail genometyping (FIG. 2C), respectively. The present invention also detected the expression of NRIP mRNA in the testis, heart and skeletal muscle tissues. The results showed that the exon2 deleted NRIP was detected by the designed F1-R primers and was not detected by the designed F2-R primers (FIG. 2D). The expression of NRIP protein in testis and skeletal muscle tissue was also performed by Western blot, in this result, the NRIP was expressed in the wild-type mouse testis and skeletal muscle tissues but not in NRIP-null mouse testis and skeletal muscle tissues (FIG. 2E).

### Example 3

### Expression of NRIP and Slow Myosin in Skeletal Muscle of Adult Male Mice

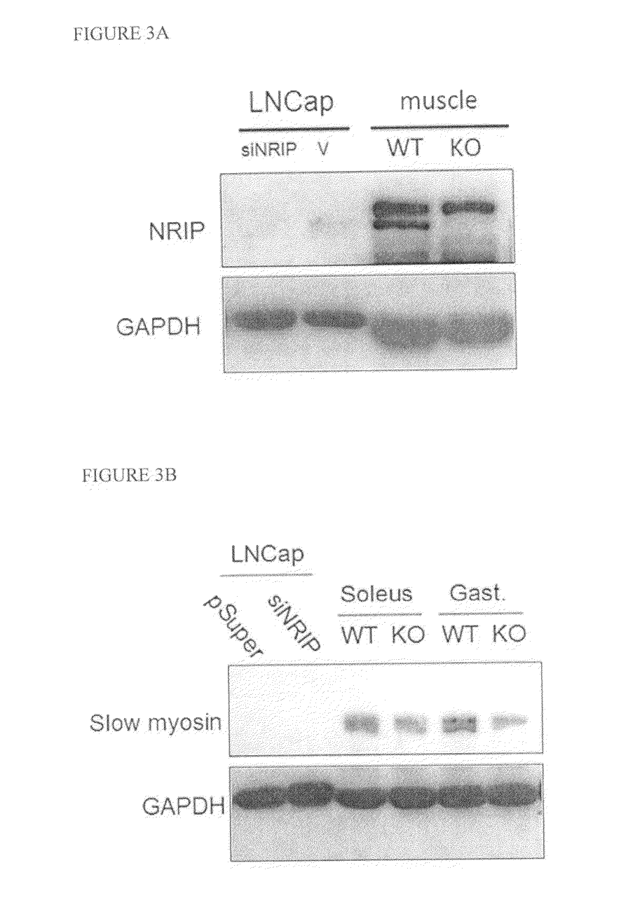

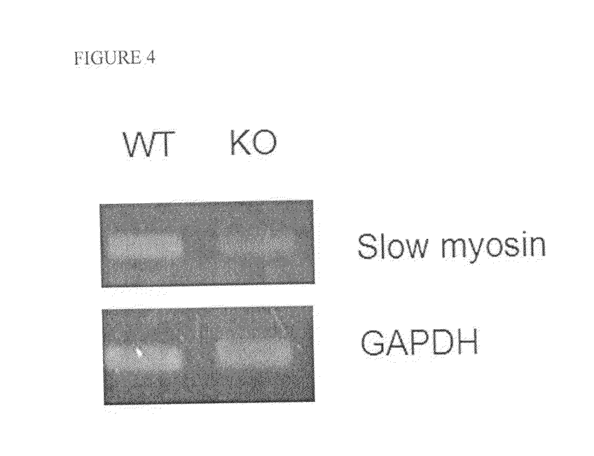

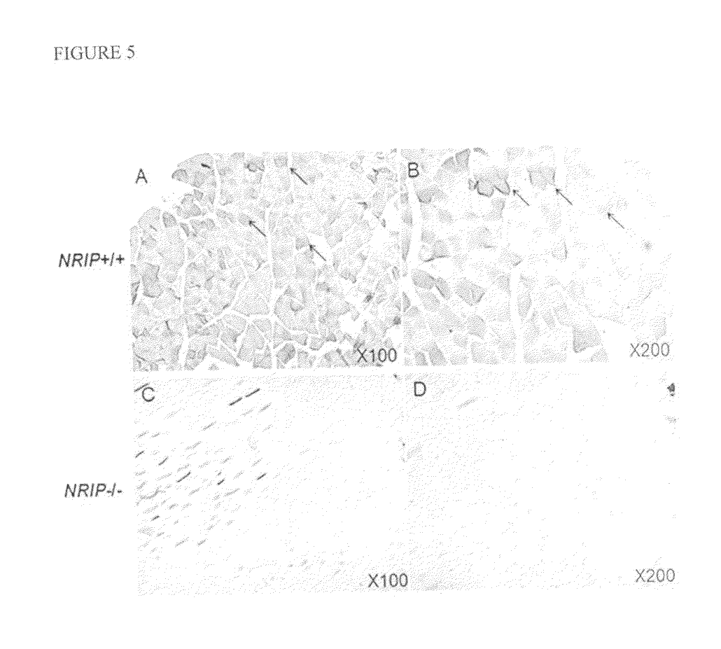

The previous results showed that the NRIP can bind to CaM. Besides, the expression of slow myosin was controlled by the Ca²⁺/CaM signaling pathway. Hence, the present invention next investigated the expression of slow myosin in NRIP wild-type and null mice. The present invention dissected the mouse soleus and gastrocnomius muscle tissue and the protein was extracted by RIPA buffer. The slow myosin and NRIP protein expression was performed by the Western blot. The results showed that the expression of slow myosin was decreased in NRIP null mice (FIG. 3B). The expression of NRIP mRNA was also decreased in NRIP null mice (FIG. 4). Moreover, the present invention also examined the expression of NRIP protein in gastrocnomius skeletal muscle tissues by IHC analysis, the result showed that the expression of NRIP was dramatically decreased in NRIP null mice (FIG. 5).

### Example 4

### Protein Expression of Slow Myosin in NRIP Overexpressed and Downregulated in C2C12 Myotube

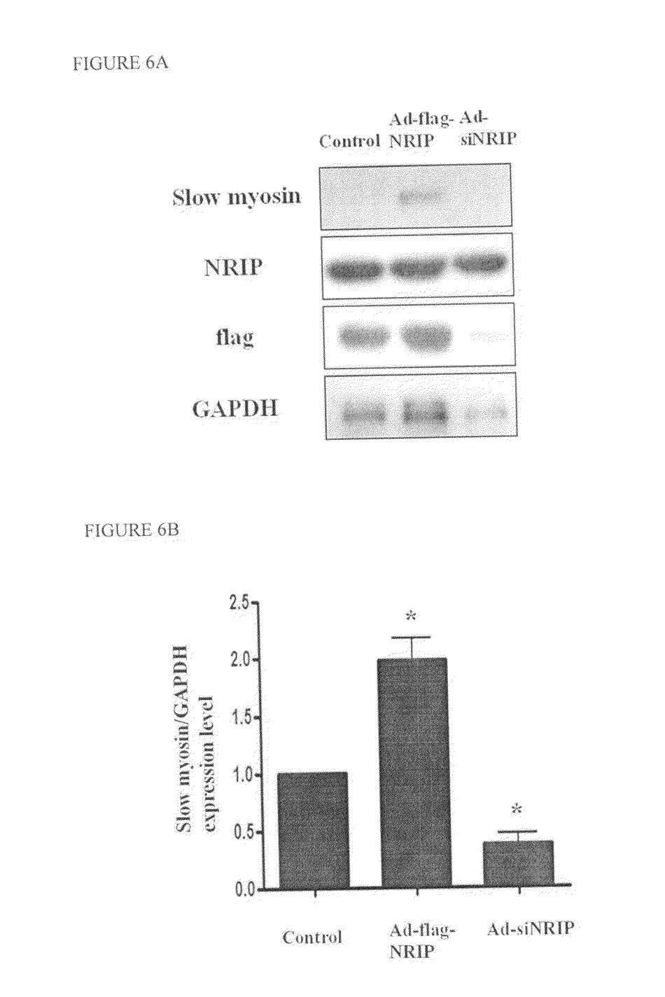

The 1×10⁵ C2C12 myoblasts were seeded in 6-well culture dish and differentiate 3 days in 5% horse serum. The adenoviral vectors (MOI 10) including control adeno-siLuciferase, adeno-flag-NRIPand adeno-siNRIP were infected into differentiated 3 days C2C12 cells. The protein were harvested in post-infected 3 days C2C12 myotube and subjected to Western blot analysis. The expression of slow myosin and endogenous NRIP were detected by anti-slow myosin and anti-NRIP primary antibody. The overexpressed flag-NRIP was detected by anti-flag primary antibody. GAPDH was the loading control (FIG. 6A). Results showed that the expression of slow myosin was downregulated to 0.5 fold when NRIP expression decreased by ad-siNRIP. However, the expression of slow myosin was upregulated to 1.9 fold in the flag-NRIP overexpressed C2C12 myotubes (FIG. 6B). Results showed that the NRIP could increase the expression of slow myosin.

While the invention has been described and exemplified in sufficient detail for those skilled in this art to make and use it, various alternatives, modifications, and improvements should be apparent without departing from the spirit and scope of the invention.

What is claimed is:

1. A method for modulating an expression level of a gene encoding slow myosin in a subject in need thereof, comprising administering to said subject a pharmaceutically effective amount of a nuclear receptor interaction protein (NRIP) and a pharmaceutically acceptable carrier.

2. The method of claim 1, wherein the NRIP interacts with a calmodulin.

3. The method of claim 1, wherein the subject is a mammal.

4. The method of claim 1, wherein the subject is a human.

5. A method for modulating an expression level of a gene encoding slow myosin in a subject in need thereof, comprising administering to said subject a pharmaceutically effective amount of an expression vector comprising a gene encoding a nuclear receptor interaction protein (NRIP) and a pharmaceutically acceptable carrier.

6. The method of claim 5, wherein the expression vector is an adenoviral vector.

7. The method of claim 5, wherein the gene is SEQ ID NO: 1.

8. The method of claim 5, wherein the NRIP interacts with a calmodulin.

9. The method of claim 5, wherein the subject is a mammal.

10. The method of claim 5, wherein the subject is a human.


SEQUENCE LISTING (6 sequences)

SEQ ID NO: 1 - DNA, 2580 bp, Mus musculus
atgtctcggggtggctcctacccacacctgttgtgggacgtgaggaaaaggtccctcgggctggaggacccgtcccggctgcggagtcgctacctgggaagaagagaatttatccaaagattaaaacttgaagcaacccttaatgtgcatgatggttgtgttaatacaatctgttggaatgacactggagaatatattttatctggctcagatgacaccaaattagtaattagtaatccttacagcagaaaggttttgacaacaattcgttcagggcaccgagcaaacatatttagtgcaaagttcttaccttgtacaaatgataaacagattgtatcctgctctggagatggagtaatattttataccaacgttgagcaagatgcagaaaccaacagacaatgccaatttacgtgtcattatggaactacttatgagattatgactgtacccaatgacccttacacttttctctcttgtggtgaagatggaactgttaggtggtttgatacacgcatcaaaactagctgcacaaaagaagattgtaaagatgatattttaattaactgtcgacgtgctgccacgtctgttgctatttgcccaccaataccatattaccttgctgttggttgttctgacagctcagtacgaatatatgatcggcgaatgctgggcacaagagctacagggaattatgcaggtcgagggactactggaatggttgcccgttttattccttcccatcttaataataagtcctgcagagtgacatctctgtgttacagtgaagatggtcaagagattctcgttagttactcttcagattacatatatctttttgacccgaaagatgatacagcacgagaacttaaaactccttctgcggaagagagaagagaagagttgcgacaaccaccagttaagcgtttgagacttcgtggtgattggtcagatactggacccagagcaaggccggagagtgaacgagaacgagatggagagcagagtcccaatgtgtcattgatgcagagaatgtctgatatgttatcaagatggtttgaagaagcaagtgaggttgcacaaagcaatagaggacgaggaagatctcgacccagaggtggaacaagtcaatcagatatttcaactcttcctacggtcccatcaagtcctgatttggaagtgagtgaaactgcaatggaagtagatactccagctgaacaatttcttcagccttctacatcctctacaatgtcagctcaggctcattcgacatcatctcccacagaaagccctcattctactcctttgctatcttctccagacagtgaacaaaggcagtctgttgaggcatctggacaccacacacatcatcagtctgataacaataatgaaaagctgagccccaaaccagggacaggtgaaccagttttaagtttgcactacagcacagaaggaacaactacaagcacaataaaactgaactttacagatgaatggagcagtatagcatcaagttctagaggaattgggagccattgcaaatctgagggtcaggaggaatctttcgtcccacagagctcagtgcaaccaccagaaggagacagtgaaacaaaagctcctgaagaatcatcagaggatgtgacaaaatatcaggaaggagtatctgcagaaaacccagttgagaaccatatcaatataacacaatcagataagttcacagccaagccattggattccaactcaggagaaagaaatgacctcaatcttgatcgctcttgtggggttccagaagaatctgcttcatctgaaaaagccaaggaaccagaaacttcagatcagactagcactgagagtgctaccaatgaaaataacaccaatcctgagcctcagttccaaacagaagccactgggccttcagctcatgaagaaacatccaccagggactctgctcttcaggacacagatgacagtgatgatgacccagtcctgatcccaggtgcaaggtatcgagcaggacctggtgatagacgctctgctgttgcccgtattcaggagttcttcagacggagaaaagaaaggaaagaaatggaagaattggatactttgaacattagaaggccgctagtaaaaatggtttataaaggccatcgcaactccaggacaatgataaaagaagccaatttctggggtgctaactttgtaatgagtggttctgactgtggccacattttcatctgggatcggcacactgctgagcatttgatgcttctggaagctgataatcatgtggtaaactgcctgcagccacatccgtttgacccaattttagcctcatctggcatagattatgacataaagatctggtcaccattagaagagtcaaggatttttaaccgaaaacttgctgatgaagttataactcgaaacgaactcatgctggaagaaactagaaacaccattacagttccagcctctttcatgttgaggatgttggcttcacttaatcatatccgagctgaccggttggagggtgacagatcagaaggctctggtcaagagaatgaaaatgaggatgaggaa

SEQ ID NO: 2 - DNA, 22 bp, Artificial Sequence
  AU primer
aggtagatttctgagtttgagg

SEQ ID NO: 3 - DNA, 25 bp, Artificial Sequence
  KU primer
gcttactttcatttatccctctttg

SEQ ID NO: 4 - DNA, 23 bp, Artificial Sequence
  XD primer
gacattcttatcagctacactag

SEQ ID NO: 5 - protein, 876 aa, Mus musculus
MARSGSCPHLLWDVRKRSLGLEDPSRLRSRYLGRREFIQRLKLEATLNVHDGCVNTICWNDTGEYILSGSDDTKLVISNPYSRKVLTTIRSGHRANIFSAKFLPCTDDKQIVSCSGDGVIFYTNIEQDAETNRQCQFTCHYGTTYEIMTVPNDPYTFLSCGEDGTVRWFDTRIKTSCTKEDCKDDILINCRRAATSVAICPPVPYYLAVGCSDSSVRIYDRRMLGTRATGNYAGRGTTGMVARFIPSHLSNKSCRVTSLCYSEDGQEILVSYSSDYIYLFDPKDDTARELKTPSAEERREELRQPPVKRLRLRGDWSDTGPRARPESERERDGEQSPNVSLMQRMSDMLSRWFEEASEVAQSNRGRGRPRPRGGTNQPDVSTLPTVPSSPNLEVCETAMDVDMPAALLQPSTSSTDPVQAQAATAAIESPRSSSLLSCPDSEPRQSVEASGHHAHHQSDNSNERLSPKPGTGEPVLSLHYSTEGTTTSTIKLNFTDEWSSTASSSRGNGSHCKSEGQEECLVPPSSVQPPEGDSETRAPEELSEKGTLPENLTQNQIDTAQLDNFPAEPLDSNSGEKNNPSQDSPCGLPEEGTLSETDRETCEQASTESATRHASTKPELPSQTEAIEQASTESATRHTSANPELPSQTEAIAPLAHEDPSARDSALQDTDDSDDDPVLIPGARYRTGPGDRRSAVARIQEFFRRRKERKEMEELDTLNIRRPLVKMVYKGHRNSRTMIKEANFWGANFVMSGSDCGHIFIWDRHTAEHLMLLEADNHVVNCLQPHPFDPILASSGIDYDIKIWSPLEESRIFNRKLADEVITRNELMLEETRNTITVPASFMLRMLASLNHIRADRLEGDRSEGSGQENENEDEE

SEQ ID NO: 6 - protein, 23 aa, Mus musculus
DRRSAVARIQEFFRRRKERKEME